(19) United States
(12) Patent Application Publication
(10) Pub. No.: US 2022/0008231 A1
(43) Pub. Date: Jan. 13, 2022

(54) STENT HAVING A TACKY SILICONE COATING TO PREVENT STENT MIGRATION

(71) Applicant: BOSTON SCIENTIFIC SCIMED, INC., Maple Grove, MN (US)

(72) Inventors: Claude O. Clerc, Marlborough, MA (US); Gerald Fredrickson, Westford, MA (US); Jonathan Root, Townsend, MA (US)

(73) Assignee: BOSTON SCIENTIFIC SCIMED, INC., Maple Grove, MN (US)

(21) Appl. No.: 17/482,060

(22) Filed: Sep. 22, 2021

Related U.S. Application Data
(63) Continuation of application No. 14/012,469, filed on Aug. 28, 2013, now abandoned.
(60) Provisional application No. 61/718,288, filed on Oct. 25, 2012.

(51) Int. Cl. A61F 2/848

(52) U.S. Cl. CPC A61F 2/848

(57) ABSTRACT

A stent having an inner surface and an outer surface, at least a portion of the outer surface of the stent comprising a tacky biocompatible coating comprising a tacky polymer material and to methods of delivering and deploying a stent using a tacky biocompatible coating comprising a tacky polymer material.

### CROSS-REFERENCE TO RELATED APPLICATIONS

This application is a continuation of U.S. patent application Ser. No. 14/012,469, filed Aug. 28, 2013, which claims priority to U.S. Patent Provisional Application No. 61/718,288, filed Oct. 25, 2012, the entire contents of which are hereby incorporated herein by reference.

### BACKGROUND OF THE INVENTION

The present invention relates generally to coated medical devices, in particular, to covered stents and to methods of using a tacky polymeric material in a patient's body lumen to prevent stent migration from a treatment site.

Stents, grafts, stent-grafts, vena cava filters and similar implantable medical devices, collectively referred to hereinafter as stents, are radially expandable or self-expanding endoprostheses which are intravascular or endoscopic implants capable of being implanted transluminally either percutaneously or endoscopically. Stents may be implanted in a variety of body lumens or vessels such as within the vascular system, urinary tracts, bile ducts, gastro-intestinal tract, airways, etc. Stents may be used to reinforce body vessels and to prevent restenosis following angioplasty in the vascular system. They may be self-expanding, mechanically expandable or hybrid expandable. In general, self-expanding stents are mounted on a delivery device consisting of two tubes. The stent is delivered by sliding the outer tube to release the stent.

Stents are typically tubular members that are radially expandable from a reduced diameter configuration for delivery through a patient's body lumen to an expanded configuration once deployed at the treatment site.

Stents may be constructed from a variety of materials such as stainless steel, Elgiloy, nickel, titanium, nitinol, polymers, shape memory polymers, etc.

Typically, the stent is formed from a tubular member in which a pattern is subsequently formed by etching or cutting material from the tubular member or it is made from wires using techniques such as braiding, knitting or weaving.

Desirable stent properties thus include sufficient flexibility to be able to conform to the tortuous body lumen during delivery, yet sufficiently rigid to resist migration once deployed at the treatment site.

In some stents, the compressible and flexible properties that assist in stent delivery may also result in a stent that has a tendency to migrate from its originally deployed position.

Stent migration affects many endoscopic stents including esophageal, duodenal, colonic, pancreatic, biliary and airway stents. It is thus desirable to provide a stent configuration that resists migration following deployment.

Commonly assigned US Patent Publication No. 20090098176, the entire content of which is incorporated by reference herein, discloses medical devices with triggerable bioadhesives.

Moreover, fully covered stents prevent tissue ingrowth and are easier to remove than bare or partially covered stents. However, these stents are even more prone to migration.

It is thus desirable to provide a stent configuration that resists migration following deployment.

Many techniques have been developed to prevent stent migration including adding barbs and flares to the stent itself or using clips or sutures to attach the stent to the vessel wall.

There remains a need in the art for an improved stent that is resistant to migration.

### SUMMARY OF THE INVENTION

In one embodiment, the present invention relates to a stent, the stent having an inner surface and an outer surface, at least a portion of the outer surface of the stent including a tacky biocompatible coating comprising a tacky silicone.

In another embodiment, the present invention relates to a stent, the stent having an inner surface and an outer surface, at least a portion of the outer surface of the stent including a tacky biocompatible coating comprising a tacky polymer material having a peel adhesion of about 20 to about 50 grams per inch, the tacky polymer having a tackiness that is not compromised by the presence of moisture.

In another embodiment, the present invention relates to a method of delivering a stent to a body lumen, the method including depositing a tacky biocompatible polymer material at a treatment site in a body lumen, delivering the stent to the treatment site and deploying the stent at the treatment site, wherein the tacky biocompatible polymer material hinders migration of the stent from the treatment site.

These and other aspects, embodiments and advantages of the present disclosure will become immediately apparent to those of ordinary skill in the art upon review of the Detailed Description and Claims to follow.

### BRIEF DESCRIPTION OF THE DRAWINGS

FIG. 1 is a side view of an embodiment of a stent according to the invention.

FIG. 2 is a cross-sectional view taken at 2 in FIG. 1 illustrating the tacky polymeric coating on the stent according to the invention.

FIG. 3 is a cross-sectional view of an alternative embodiment of a stent similar to that shown in FIG. 1 having the tacky polymeric coating and a hydrophilic coating or biodegradable coating disposed on the tacky polymeric coating.

FIG. 4 is a cross-sectional view of a covered stent according to the invention.

### DETAILED DESCRIPTION OF THE INVENTION

While embodiments of the present invention may take many forms, there are described in detail herein, specific embodiments of the present disclosure. This description is an exemplification of the principles of the present disclosure and is not intended to limit the disclosure to the particular embodiments illustrated herein.

The present invention is directed to implantable medical devices such as stents having a tacky coating thereon to prevent stent migration and to methods of using a tacky polymeric material in a patient's body lumen to prevent stent migration from a treatment site.

The term “tacky” is a well know term in the adhesives art. As used herein, the term “tacky” shall refer to a material that retains a sticky or slightly sticky feel to the touch. These materials can also be referred to as pressure sensitive polymer materials. The tacky materials employed herein can have peel strengths from about 20 grams/inch to about 1000 grams/inch as measured per ASTM D3330 Standard Test Method for Peel Adhesion of Pressure-Sensitive Tape, suitably about 20 grams/inch to about 500 grams/inch, and more suitably about 20 grams/inch to about 100 grams/inch. In some embodiments, the tacky materials employed herein have a low peel strength of about 20 grams/inch to about 50 grams/inch.

The tacky polymeric materials employed herein are selected to as to provide gentle adhesion to human tissue. However, the adhesion is not permanent and the materials can be readily removed when desired.

It is also desirable that the tackiness or adhesion of the tacky polymeric material is not compromised upon exposure to moisture such as would be the case upon insertion in a patient's body.

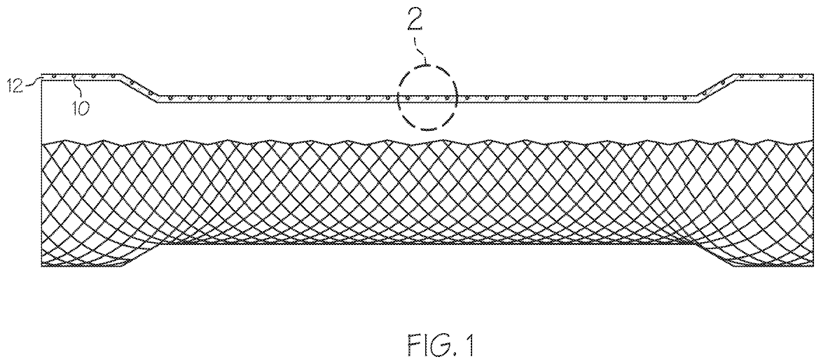

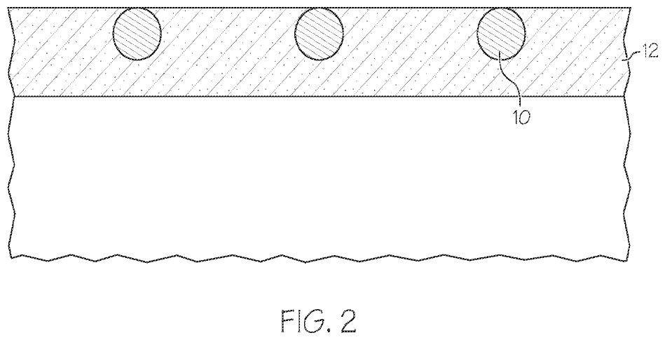

Turning now to the drawings, FIG. 1 is a side view of one embodiment of a stent on which the coatings according to the invention be employed. In this embodiment, stent 10 is a self-expanding stent formed of a shape memory metal such as nitinol having a silicone covering. The stent has a braided wire construction. In this embodiment, stent 10 is shown having a silicone covering 12. Stent 10 is disposed on silicone covering 12 and is partially embedded therein. FIG. 2 is a partial cross-sectional view of the stent taken at section 2 in FIG. 1. Stents of this type are described in commonly assigned US Patent Publication Nos. 2006/0276887 and 2008/0009934, each of which is incorporated by reference herein in its entirety.

While in the embodiment shown in FIGS. 1 and 2, the stent is formed from nitinol, stents may be constructed of any suitable stent material including, but not limited to stainless steel, Elgiloy, nickel, titanium, nitinol, shape memory polymers, other polymeric materials, etc.

Any stent can have a covering and the coverings are thus not limited to nitinol stents. Moreover, the stent need not be covered whatsoever, may be partially covered or may be fully covered.

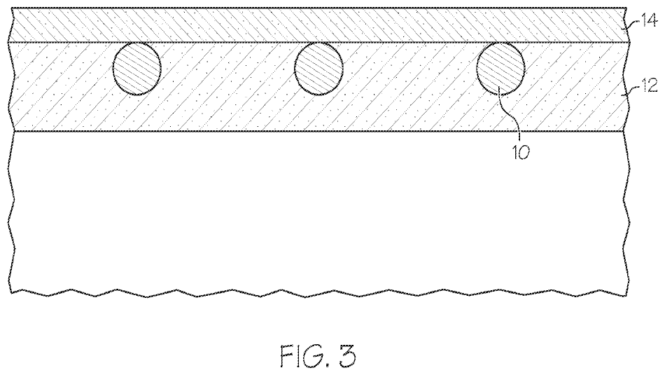

Other suitable covering materials can be employed as well. Examples of other suitable covering materials include, but are not limited to, polyethylene, polypropylene, polyvinyl chloride, polytetrafluoroethylene, including expanded polytetrafluoroethylene (ePTFE), fluorinated ethylene propylene, fluorinated ethylene propylene, polyvinyl acetate, polystyrene, poly(ethylene terephthalate), naphthalene, dicarboxylate derivatives, such as polyethylene naphthalate, polybutylene naphthalate, polytrimethylene naphthalate and trimethylenediol naphthalate, polyurethane, polyurea, polyamides, polyimides, polycarbonates, polyaldehydes, polyether ether ketone, natural rubbers, polyester copolymers, styrene-butadiene copolymers, polyethers, such as fully or partially halogenated polyethers, and copolymers and combinations thereof. See, for example, commonly assigned U.S. Pat. No. 8,114,147, the entire content of which is incorporated by reference herein. Stent 10 further has a tacky coating as shown in cross-section in FIG. 3. In this embodiment, tacky coating 14 comprises a tacky silicone.

Tacky or pressure sensitive silicone materials are commercially available from a variety of sources such as MED 6300 series of heat cured silicone materials and MED 6381 moisture cured silicone available from Nusil located in Santa Barbara, CA.

In some embodiments, the tacky silicone is a moisture cured silicone.

In some embodiments, the tacky silicone gel is a polydimethylsiloxane.

While tacky silicone is one desirable tacky polymeric material that may be employed herein, other tacky polymeric materials may be used as well including, but not limited to, styrenic block copolymers such as styrene-isobutylene-styrene (SIBS), styrene-ethylene/butylene-styrene (SEBS), styrene-ethylene/propylene-styrene (SEPS) and styrene-isoprene-styrene (SIS), acrylics, polyvinyl ether, polyurethanes, copolymers of ethylene such as ethylene vinyl acetate (EVA), etc.

The silicone gels have a similar feel to that of a hydrogel. However, the tackiness or adhesion of a hydrogel is compromised in the presence of moisture. Hydrogels are known to become “slippery” when wet, making them ideally suited for delivery of medical devices in a patient's body lumen where lubricity is desirable.

In some embodiments, a biocompatible dye is added to the tacky polymeric material to make it readily visible to a physician.

The tacky polymeric material may be applied to the entire outer surface of the stent, or to portions of the stent such as to the distal, proximal and central portions of the stent.

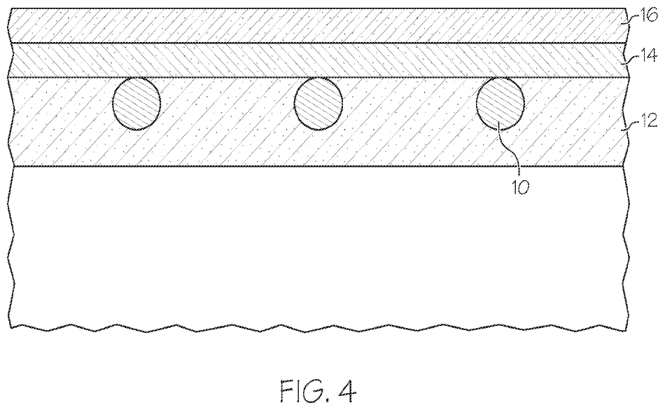

If the tacky polymeric material is applied to the stent, it may be desirable to dispose a hydrophilic or biodegradable coating over the tacky polymeric material to facilitate delivery through a patient's body lumen for a balloon expandable stent or to decrease the friction force between the outer tube of the delivery device and the stent for a self-expanding stent. FIG. 4 is a cross-sectional view of a stent 10 including a covering 12, a tacky polymeric coating 14 and a hydrophilic or biodegradable coating 16 disposed on the tacky polymeric coating 14. Once the stent is positioned and deployed at the treatment site, the biodegradable or hydrophilic coating will erode, exposing the underlying tacky polymeric coating which is now positioned between the patient's vessel wall and the stent in order to hinder stent migration.

Examples of suitable hydrogels include, but are not limited to, polyvinylpyrrolidone (PVP), poly(meth)acrylic acid and copolymers of (meth)acrylic acid, polyacrylate, chitosan, polyalkylene glycols such as polyethylene glycol (PEG) or polypropylene glycol, polyethylene glycol/dextran aldehyde, polyalkylene oxides such as polyethylene oxide and polypropylene oxide, polyvinyl esters such as polyvinyl acetate, polyhydroxyethyl methacrylate, polyvinyl alcohol, polyvinyl ether, and so forth. High molecular weight starches and carbohydrates may also be employed.

Hydrogel materials are disclosed in commonly assigned U.S. Pat. No. 5,693,034 to Buscemi et al., the entire content of which is incorporated by reference herein.

Any suitable biodegradable material can be employed herein that does not form an adhesive layer. These biodegradable materials break down and lose their integrity in vivo. Examples of suitable biodegradable polymers include, but are not limited to, poly(amides) such as poly(amino acids) and poly(peptides), poly(esters) such as polylactide including poly(DL-lactide) and polyglycolide, and copolymers thereof such as polylactide-co-glycolide including poly(DL-lactide-co-glycolide), poly(L-lactide-co-glycolide), poly(caprolactone) and polylactide-co-caprolactone including poly(DL-lactide-co-caprolactone and poly(L-lactide-co-caprolactone), poly(anhydrides), poly(orthoesters), poly(carbonates) including tyrosine derived polycarbonates, polyhydroxyvalerate, polyhydroxybutyrate, polyhydroxybutyrate-co-valerate, and chemical derivatives thereof (substitutions, additions of chemical groups, for example, alkyl, alkylene, hydroxylations, oxidations, and other modifications routinely made by those skilled in the art), copolymers and mixtures thereof.

Therapeutic agents may be incorporated in the tacky polymeric material, the hydrophilic or biodegradable coating layer, or both.

Various therapeutic agents may be employed herein depending on the condition which is being treated. As used herein, the terms, “therapeutic agent”, “drug”, “pharmaceutically active agent”, “pharmaceutically active material”, “beneficial agent”, “bioactive agent”, and other related terms may be used interchangeably herein and include genetic therapeutic agents, non-genetic therapeutic agents and cells. A drug may be used singly or in combination with other drugs. Drugs include genetic materials, non-genetic materials, and cells.

A therapeutic agent may be a drug or other pharmaceutical product such as non-genetic agents, genetic agents, cellular material, etc. Some examples of suitable non-genetic therapeutic agents include but are not limited to: antithrombogenic agents such as heparin, heparin derivatives, vascular cell growth promoters, growth factor inhibitors, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof.

Other active agents include, but are not limited to, antineoplastic, antiproliferative, antimitotic, antiinflammatory, antiplatelet, anticoagulant, antifibrin, antiproliferative, antibiotic, antioxidant, and antiallergic substances as well as combinations thereof.

Examples of antineoplastic/antiproliferative/antimitotic agents include, but are not limited to, paclitaxel (e.g., TAXOL® by Bristol-Myers Squibb Co., Stamford, Conn.), the olimus family of drugs including sirolimus (rapamycin), biolimus (derivative of sirolimus), everolimus (derivative of sirolimus), zotarolimus (derivative of sirolimus) and tacrolimus, methotrexate, azathiprine, vincristine, vinblastine, 5-fluorouracil, doxorubicin hydrochloride, mitomycin, cisplatin, vinblastine, vincristine, epothilones, endostatin, angiostatin and thymidine kinase inhibitors. While the preventative and treatment properties of the foregoing therapeutic substances or agents are well-known to those of ordinary skill in the art, the substances or agents are provided by way of example and are not meant to be limiting. Other therapeutic substances are equally applicable for use with the disclosed methods and compositions. See commonly assigned U.S. Patent Application Nos. 2010/0087783, 2010/0069838, 2008/0071358 and 2008/0071350, each of which is incorporated by reference herein. See also commonly assigned U.S. Patent Application Nos. 2004/0215169 and 2009/0098176, and U.S. Pat. No. 6,805,898, each of which is incorporated by reference herein.

Derivatives of many of the above mentioned compounds also exist which are employed as therapeutic agents and of course mixtures of therapeutic agents may also be employed.

For application, the therapeutic agent can be dissolved in a solvent or a cosolvent blend along with the bioadhesive or biodegradable polymer material.

Suitable solvents include, but are not limited to, dimethyl formamide (DMF), butyl acetate, ethyl acetate, tetrahydrofuran (THF), dichloromethane (DCM), acetone, acetonitrile, dimethyl sulfoxide (DMSO), butyl acetate, etc.

In other embodiments, the tacky polymeric material is delivered to the treatment site prior to delivery and deployment of the stent, for example via injection with a syringe, as in the esophagus, through or along the scope with a catheter, providing the viscosity is not too high for injection.

In some embodiments, the tacky polymeric material is a moisture cured polydimethylsiloxane which, once delivered to the treatment site, cures in the presence of moisture.

Once cured, the stent is then delivered and deployed at the treatment site. The tacky silicone material hinders stent migration. The stent can also be delivered before the tacky polymer is fully cured.

If the stent is a removable stent, the moisture cured polydimethylsiloxane can then be removed similar to removal of a dermal patch, such as with forceps.

If the viscosity of the tacky polymeric material is too high, it can be delivered as a patch and delivered with a scope. Suitably, the patch is about 1 cm in diameter, or delivery may involve the use of several smaller patches at more than one location to coincide with several locations along the stent, for example, distal, center and proximal locations of the stent.

If a patch is employed, it may be desirable to dispose a hydrophilic or biodegradable coating on the tacky polymeric material to facilitate delivery through a patient's body lumen.

If a hydrophilic coating is employed, water can be injected into the body lumen to facilitate dissolution/removal of the hydrophilic coating.

An alternative delivery technique is to employ a balloon to delivery the tacky polymeric material to the treatment site. Either patches or tubular members of the tacky polymeric material may be delivered in this manner. Multiple tubular members can also be delivered on a single balloon if desired.

For balloon delivery, it may also be desirable to dispose a hydrophilic polymer material on the tacky polymeric material to facilitate delivery through a patient's body lumen. The hydrophilic polymer may also be applied to the inner surface of the tacky polymeric material to prevent adhesion to the balloon. Dissolution/removal of the hydrophilic material can be facilitated by injecting water into the body lumen.

These techniques are most suitably employed with stents used in the gastrointestinal tract, but the techniques are not limited as such.

The description provided herein is not to be limited in scope by the specific embodiments described which are intended as single illustrations of individual aspects of certain embodiments. The methods, compositions and devices described herein can comprise any feature described herein either alone or in combination with any other feature(s) described herein. Indeed, various modifications, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference in their entirety into the specification to the same extent as if each individual publication, patent or patent application was specifically and individually indicated to be incorporated herein by reference. Citation or discussion of a reference herein shall not be construed as an admission that such is prior art.

1. A stent device, the stent device having an inner surface and an outer surface, the stent device comprising:
a stent body;
a silicone covering disposed on at least one surface of the stent body; and
a tacky silicone material comprising polydimethylsiloxane, the tacky silicone material selected so as to provide adhesion to human tissue and disposed over the silicone covering, wherein the tacky silicone material has a peel adhesion of about 20 grams per inch to about 1000 grams per inch.

2. The stent device of claim 1 wherein the tacky silicone material has a tackiness that is not compromised by the presence of moisture.

3. The stent device of claim 1, wherein the tacky silicone material defines an entirety of the outer surface of the stent device prior to implantation of the stent device.

4. The stent device of claim 1 wherein the tacky silicone material is pressure sensitive.

5. The stent device of claim 1 wherein the peel adhesion of said tacky silicone material is about 20 grams per inch to about 100 grams per inch.

6. The stent device of claim 1 wherein the peel adhesion of said tacky silicone material is about 20 grams per inch to about 50 grams per inch.

7. The stent device of claim 1 wherein the tacky silicone material includes a biocompatible dye.

8. The stent device of claim 1 wherein the silicone covering comprises a partial or full covering of the stent body.

9. The stent device of claim 1 wherein the stent comprises a member selected from the group consisting of esophageal stents, pancreatic stents, duodenal stents, colonic stents, biliary stents and airway stents.

10. The stent device of claim 1, wherein the stent body is at least partially embedded within the silicone covering.

11. The stent device of claim 1, further comprising a biodegradable coating surrounding and disposed on an outer surface of the tacky silicone material.

12. A stent device having an inner surface and an outer surface, the stent device comprising:
a stent body;
a silicone covering disposed on at least one surface of the stent body; and
a tacky biocompatible coating comprising polydimethylsiloxane disposed over the silicone covering, the tacky biocompatible coating having a peel adhesion of about 20 grams per inch to about 50 grams per inch, the tacky biocompatible coating having a tackiness that is not compromised by the presence of moisture and is pressure sensitive, wherein said tacky biocompatible coating defines an entirety of the outer surface of the stent device prior to implantation of the stent device.

13. The stent device of claim 12, wherein the tacky biocompatible coating is selected to provide adhesion to human tissue.

14. The stent device of claim 12, wherein the stent body is at least partially embedded within the silicone covering.

15. The stent device of claim 12, wherein the tacky biocompatible coating includes a therapeutic agent.

16. The stent device of claim 12 wherein the tacky biocompatible coating includes a biocompatible dye.

17. The stent device of claim 12, wherein the silicone covering extends radially inward of the stent body.

18. The stent device of claim 12 wherein the stent comprises a member selected from the group consisting of esophageal stents, pancreatic stents, duodenal stents, colonic stents, biliary stents and airway stents.

19. The stent device of claim 12, further comprising a biodegradable coating surrounding and disposed on an outer surface of the tacky biocompatible coating.

20. A stent device, the stent device having an inner surface and an outer surface, the stent device comprising:
a stent body;
a silicone covering disposed on a surface of the stent body;
a tacky silicone material comprising polydimethylsiloxane having a peel strength of 20 grams per inch to 500 grams per inch and having a tackiness that is not compromised by the presence of moisture, the tacky silicone material surrounding and disposed on an outer surface of the silicone covering; and
a biodegradable coating surrounding and disposed on an outer surface of the tacky silicone material.
